(19) United States
(12) Patent Application Publication
(10) Pub. No.: US 2009/0292132 A1
(43) Pub. Date: Nov. 26, 2009

(54) REACTOR SYSTEM AND PROCESS FOR REACTING A FEED

(76) Inventor: Wayne Errol EVANS, Richmond, TX (US)

Correspondence Address: Lisa Kay Holthus;c/o Shell Oil Company, Intellectual Property, P.O. Box 2463, Houston, TX, 77252-2463, US

(21) Appl. No.: 12/121,691

(22) Filed: May 15, 2008

Related U.S. Application Data
(60) Provisional application No. 60/938,880, filed on May 18, 2007.

(51) Int. Cl. C07D 301/02; B01J 8/02; B01J 19/00; C07C 31/20; C07C 41/02; C07C 69/96; C07C 209/00

(52) U.S. Cl. 549/518; 422/211; 422/201; 568/867; 568/680; 558/260; 564/487

(57) ABSTRACT

A reactor system comprising:
a reactor vessel, and
positioned inside the reactor vessel, an absorbent and a catalyst positioned downstream from the absorbent;
   a process for reacting a feed; and a process for preparing a 1,2-diol, a 1,2-diol ether, a 1,2-carbonate, or an alkanolamine.

### CROSS-REFERENCE TO RELATED APPLICATIONS

This application claims the benefit of U.S. Provisional Patent Application No. 60/938,880, filed May 18, 2007, which is incorporated herein by reference in its entirety.

### FIELD OF THE INVENTION

The invention relates to a reactor system and a process for reacting a feed comprising a hydrocarbon and sulfur impurities which process utilizes the inventive reactor system.

### BACKGROUND OF THE INVENTION

Industrial-scale preparations of hydrocarbons yield impure hydrocarbons. Typically, the hydrocarbons are subjected to a purification process to reduce the impurities. However, low levels of impurities still remain in the hydrocarbons and can act as catalyst poisons in a subsequent process, adversely affecting the performance of the catalyst. Of particular concern are trace sulfur impurities that may be present in the hydrocarbons. Certain processes react a feed comprising a hydrocarbon with a metal or noble metal catalyst. These catalysts are generally susceptible to sulfur poisoning since many metals are known to form sulfides even if sulfur is present in the feed in quantities below the parts per million level. Processes using metal or noble metal catalysts susceptible to sulfur poisoning include, but are not limited to, ammoxidation reactions, dehydrogenation reactions, catalytic reforming reactions, and oxidation reactions, in particular partial oxidation of an olefin to form an olefin oxide such as ethylene oxide. These reactions are typically highly exothermic and generally performed in a vertical shell-and-tube heat exchanger comprising a multitude of reaction tubes, each containing a packed bed of solid particulate catalyst and surrounded by a heat exchange fluid. In the production of olefin oxides, such as ethylene oxide, silver-based catalysts are used to convert ethylene and oxygen into ethylene oxide. These silver-based catalysts are especially susceptible to sulfur poisoning even at sulfur quantities on the order of parts per billion levels. The catalyst poisoning impacts the catalyst performance, in particular the selectivity or activity, and shortens the length of time the catalyst can remain in the reactor before having to exchange the poisoned catalyst with fresh catalyst.

Typical sulfur impurities present in the hydrocarbons such as olefins include, but are not limited to, dihydrogen sulfide, carbonyl sulfide, mercaptans, and organic sulfides. Mercaptans and organic sulfides, especially organic sulfides, are particularly difficult sulfur impurities to remove from the feed. Additional impurities may include, acetylene, carbon monoxide, phosphorous, arsenic, selenium, and halogens. An olefin such as ethylene may be derived from several sources including, but not limited to, petroleum processing streams such as those generated by a thermal cracker, a catalytic cracker, a hydrocracker or a reformer, natural gas fractions, naphtha and organic oxygenates such as alcohols.

Over the years, much effort has been devoted to improving the olefin epoxidation process. Solutions have been found in various improved reactor designs.

For example, U.S. Pat. No. 6,939,979 describes the use of an alkali metal treated inert as a diluent for the catalyst positioned in an upper section of the reactor tubes. Treating the inert with an alkali metal reduces the degradation of ethylene oxide by the inert thereby improving the selectivity to ethylene oxide. However, placing an inert material upstream from the catalyst does not significantly reduce the amount of sulfur-containing impurities present in the feed which can poison the catalyst.

Thus, not withstanding the improvements already achieved, there exists a desire for a reactor system and reaction process that further improves the performance of the catalyst, in particular the duration of time the catalyst remains in the reactor before exchanging with a fresh catalyst.

### SUMMARY OF THE INVENTION

The present invention provides an epoxidation reactor system comprising:

an epoxidation reactor vessel, and

positioned inside the epoxidation reactor vessel, an absorbent comprising a metal having an atomic number of 22 through 44 or 82 and an epoxidation catalyst positioned downstream from the absorbent.

The invention also provides a process for reacting a feed comprising an olefin, oxygen, and one or more impurities, which process comprises:

contacting the feed with an absorbent comprising a metal having an atomic number of 22 through 44 or 82 positioned within an epoxidation reactor system according to the present invention to reduce the quantity of the one or more impurities in the feed; and

subsequently contacting the feed with an epoxidation catalyst to yield an olefin oxide.

Further, the invention provides a process of preparing a 1,2-diol, a 1,2-diol ether, a 1,2-carbonate, or an alkanolamine comprising obtaining an olefin oxide by the process according to this invention, and converting the olefin oxide into the 1,2-diol, the 1,2-diol ether, the 1,2-carbonate, or the alkanolamine.

### BRIEF DESCRIPTION OF THE DRAWINGS

FIG. 1 is a schematic view of a reactor system according to an embodiment of the invention which has the absorbent positioned inside the reactor tubes.

FIG. 2 is a schematic view of a reactor system according to an embodiment of the invention which has the absorbent positioned inside the reactor vessel and upstream from the reactor tubes.

### DETAILED DESCRIPTION OF THE INVENTION

In accordance with this invention, an epoxidation reactor system is provided comprising an epoxidation reactor vessel, an absorbent and an epoxidation catalyst. The absorbent and the catalyst are positioned inside the reactor vessel with the catalyst positioned downstream from the absorbent. Absorbents have been used for purifying hydrocarbons for many years. An important aspect of this invention is the recognition only after many years that an absorbent can be used in an epoxidation reactor vessel to reduce the amount of impurities in the feed, in particular sulfur impurities. It is unexpected that the absorbent can reduce the impurities in the feed under the conditions experienced inside the reactor vessel. It is also an unexpected advantage of the present invention that the impurities can be reduced in the feed without requiring any additional equipment such as an auxiliary vessel or pipe containing the absorbent.

The terms “substantially vertical” and “substantially horizontal”, as used herein, are understood to include minor deviations from true vertical or horizontal positions relative to the central longitudinal axis of the reactor vessel, in particular the terms are meant to include variations ranging from 0 to 20 degrees from true vertical or horizontal positions. True vertical is aligned along the central longitudinal axis of the reactor vessel. True horizontal is aligned perpendicular to the central longitudinal axis of the reactor vessel.

The term “substantially parallel”, as used herein, is understood to include minor deviations from a true parallel position relative to the central longitudinal axis of the reactor vessel, in particular the term is meant to include variations ranging from 0 to 20 degrees from a true parallel position relative to the central longitudinal axis of the reactor vessel.

Referring now to preferred embodiments of the invention, the epoxidation reactor vessel of the present invention may be any reactor vessel used to react a feed containing an olefin and oxygen. The reactor vessel may contain one or more open-ended reactor tubes. Preferably, the reactor vessel may contain a plurality of reactor tubes. The reactor tubes may be any size. Suitably, a reactor tube may have an internal diameter of at least 5 mm (millimeters), in particular at least 10 mm.

Preferably, the epoxidation reactor vessel is a shell-and-tube heat exchanger containing a plurality of reactor tubes. The reactor tubes may preferably have an internal diameter in the range of from 15 to 80 mm, more preferably from 20 to 75 mm, and most preferably from 25 to 70 mm. The reactor tubes may preferably have a length in the range of from 5 to 20 m (meters), more preferably from 10 to 15 m. The shell-and-tube heat exchanger may contain from 1000 to 20000 reactor tubes, in particular from 2500 to 15000 reactor tubes.

The one or more reactor tubes are positioned substantially parallel to the central longitudinal axis of the reactor vessel and are surrounded by a shell adapted to receive a heat exchange fluid (i.e., the shell side of the shell-and-tube heat exchanger). The heat exchange fluid in the heat exchange chamber may be any fluid suitable for heat transfer, for example water or an organic material suitable for heat exchange. The organic material may include oil or kerosene. The upper ends of the one or more reactor tubes are connected to a substantially horizontal upper tube plate and are in fluid communication with the one or more inlets to the reactor vessel, and the lower ends of the one or more reactor tubes are connected to a substantially horizontal lower tube plate and are in fluid communication with the one or more outlets to the reactor vessel (i.e., the tube side of the shell-and-tube heat exchanger). The reactor vessel contains a packed bed of absorbent. The absorbent may be positioned inside the one or more reactor tubes and/or upstream from the one or more reactor tubes, for example positioned on top of the upper tube plate and reactor tubes in the headspace of the reactor vessel. Preferably, the absorbent may be positioned inside the one or more reactor tubes.

When the absorbent is placed inside the one or more reactor tubes, the absorbent may have a bed height of at least 0.25% of the length of the reactor tube, in particular at least 0.5%, more in particular at least 1%, most in particular at least 2% of the length of the reactor tube. When the absorbent is placed inside the one or more reactor tubes, the absorbent may have a bed height of at most 20% of the length of the reactor tube, in particular at most 15%, more in particular at most 10%, most in particular at most 5% of the length of the reactor tube.

When the absorbent is positioned upstream from the one or more reactor tubes, the absorbent may have a bed height of at least 0.05 m, in particular at least 0.075 m, more in particular at least 0.1 m, most in particular at least 0.15 m. When the absorbent is positioned upstream from the one or more reactor tubes, the absorbent may have a bed height of at most 2 m, in particular at most 1 m, more in particular at most 0.5 m.

The one or more reactor tubes contain a packed bed of catalyst positioned downstream from the absorbent. In the normal practice of this invention, a major portion of the catalyst bed comprises catalyst particles. By a “major portion” it is meant that the ratio of the weight of the catalyst particles to the weight of all the particles contained in the catalyst bed is at least 0.50, in particular at least 0.8, preferably at least 0.85, more preferably at least 0.9. Particles which may be contained in the catalyst bed other than the catalyst particles are, for example, inert particles; however, it is preferred that such other particles are not present in the catalyst bed. The catalyst bed is supported in the one or more reactor tubes by a catalyst support means arranged in the lower ends of the reactor tubes. The support means may include a screen or a spring.

The one or more reactor tubes may also contain a separate bed of particles of an inert material for the purpose of, for example, heat exchange with a feedstream. Such separate bed may be used especially when the absorbent bed is positioned upstream from the one or more reactor tubes. The one or more reactor tubes may also contain another such separate bed of inert material for the purpose of, for example, heat exchange with the reaction product. Alternatively, rod-shaped metal inserts may be used in place of the bed of inert material. For further description of such inserts, reference is made to U.S. Pat. No. 7,132,555, which is incorporated by reference.

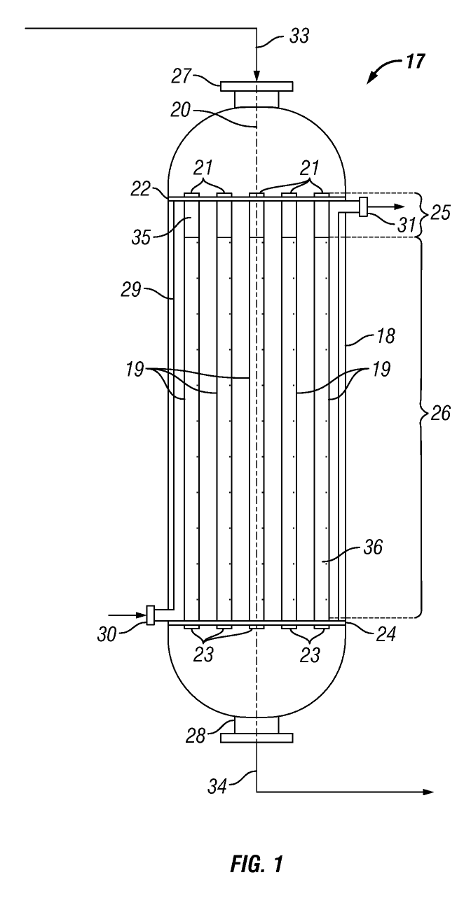

Reference is made to FIG. 1, which is a schematic view of an epoxidation reactor system (17) comprising a shell-and-tube heat exchanger reactor vessel having a substantially vertical vessel (18) and a plurality of open-ended reactor tubes (19) positioned substantially parallel to the central longitudinal axis (20) of the epoxidation reactor vessel (18). The upper ends (21) of the reactor tubes (19) are connected to a substantially horizontal upper tube plate (22) and the lower ends (23) of the reactor tubes (19) are connected to a substantially horizontal lower tube plate (24). The upper tube plate (22) and the lower tube plate (24) are supported by the inner wall of the reactor vessel (18). The plurality of reactor tubes (19) contain an absorbent bed (25) and a catalyst bed (26) positioned downstream from the absorbent bed. The absorbent bed (25) contains an absorbent (35). The catalyst bed (26) contains an epoxidation catalyst (36). The catalyst bed (26) is supported in the reactor tubes (19) by a catalyst support means (not shown) arranged in the lower ends (23) of the reactor tubes (19). Components of the feed (33), such as the olefin and oxygen, enter the reactor vessel (18) via one or more inlets such as inlet (27) which are in fluid communication with the upper ends (21) of the reactor tubes (19). The reaction product (34) exits the epoxidation reactor vessel (18) via one or more outlets such as outlet (28) which are in fluid communication with the lower ends (23) of the reactor tubes (19). The heat exchange fluid enters the heat exchange chamber (29) via one or more inlets such as inlet (30) and exits via one or more outlets such as outlet (31). The heat exchange chamber (29) may be provided with baffles (not shown) to guide the heat exchange fluid through the heat exchange chamber (29).

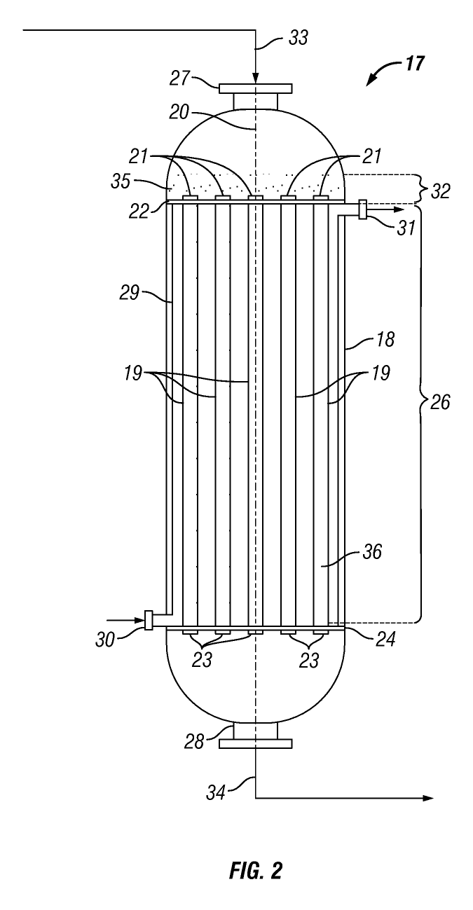

FIG. 2 is a schematic view of an epoxidation reactor system (17) comprising a shell-and-tube heat exchanger reactor vessel (18) similar to FIG. 1 except that the absorbent bed (32) is positioned upstream from the reactor tubes (19).

The present invention also provides a process for reacting a feed comprising an olefin, oxygen, and one or more impurities by contacting the feed with an absorbent positioned within an epoxidation reactor vessel, reducing the quantity of the one or more impurities in the feed; and subsequently contacting the feed with an epoxidation catalyst which is positioned within the epoxidation reaction vessel downstream from the absorbent, yielding a reaction product comprising an olefin oxide. The term “reaction product” as used herein is understood to refer to the fluid exiting from the outlet of the reactor vessel.

Typically, the temperature of the absorbent may be at least 130° C., in particular at least 140° C., more in particular at least 150° C. The temperature of the absorbent may be at most 350° C., in particular at most 320° C., more in particular at most 300° C. The temperature of the absorbent may be in the range of from 150 to 320° C., preferably from 180 to 300° C., most preferably from 210 to 270° C.

The reaction temperature in the reaction zone containing the epoxidation catalyst may be at least 130° C., in particular at least 150° C., more in particular at least 180° C., most in particular at least 200° C. The reaction temperature may be at most 350° C., in particular at most 325° C., more in particular at most 300° C. The reaction temperature may be in the range of from 150 to 350° C., preferably from 180 to 300° C.

The absorbent comprises a metal having an atomic number of 22 through 44 or 82, in particular 22 through 30. Preferably, the absorbent comprises one or more metals selected from cobalt, chromium, copper, manganese, nickel, and zinc, in particular the one or more metals are selected from copper, nickel and zinc, more in particular the one or more metals comprise copper. Preferably, the absorbent comprises copper and one or more metals having an atomic number of 22 through 44. More preferably, the absorbent comprises copper and one or more metals selected from manganese, chromium, zinc, and combinations thereof. Most preferably, the absorbent comprises copper and zinc. The metal may be present in reduced or oxide form, preferably as an oxide. The absorbent may also contain a support material. The support material may be selected from alumina, titania, silica, activated carbon or mixtures thereof. Preferably, the support material may be alumina, in particular alpha-alumina. Without wishing to be bound by theory, it is believed the absorbent reduces the impurities in the feed by chemical or physical means including, but not limited to, reaction with the impurities and absorption of the impurities.

The absorbent may be prepared by conventional processes for the production of such metal-containing materials, for example by precipitation or impregnation, preferably by precipitation. For example, in the precipitation process, a suitable salt of copper, optional additional metal salt, and optional salt of the support material may be prepared by reacting the metals with a strong acid such as nitric acid or sulfuric acid. The resulting salts may then be contacted with a basic bicarbonate or carbonate solution in a pH range of from 6 to 9 at a temperature from 15 to 90° C., in particular 80° C., to produce a precipitate of metal oxide. The precipitate may be filtered and then washed at a temperature in the range of from 20 to 50° C. The precipitate may then be dried at a temperature in the range of from 100 to 160° C., in particular 120 to 150° C. After drying, the precipitate may then be calcined at a temperature in the range of from 170 to 600° C., in particular from 350 to 550° C. The precipitate may be formed into a desired size and shape by conventional processes such as extrusion or tableting. Alternatively, an impregnation process may be used to form the absorbent by impregnating the support material with suitable solutions of the metal compounds followed by drying and calcining.

The size and shape of the absorbent may be in the form of chunks, pieces, cylinders, rings, spheres, wagon wheels, tablets, and the like of a size suitable for employment in a fixed bed reactor vessel, for example from 2 mm to 30 mm. Preferably, the size and shape maximizes the surface area available for contact with the feed.

The absorbent after calcination may contain metal oxide in a quantity in the range of from 20 to 100% w (percent by weight), relative to the weight of the absorbent, in particular from 70 to 100% w, relative to the weight of the absorbent, more in particular from 75 to 95% w, relative to the weight of the absorbent.

The support material may be present in the absorbent after calcination in a quantity of at least 1% w, relative to the weight of the absorbent, in particular at least 1.5% w, more in particular at least 2% w, relative to the weight of the absorbent. The support material may be present in the absorbent after calcination in a quantity of at most 80% w, relative to the weight of the absorbent, in particular at most 50% w, more in particular at most 30% w, relative to the weight of the absorbent, most in particular at most 25% w, relative to the weight of the absorbent. The support material may be present in the absorbent after calcination in a quantity in the range of from 5 to 25% w, in particular from 10 to 20% w, relative to the weight of the absorbent.

When the absorbent comprises copper, the absorbent after calcination may contain copper oxide in a quantity of at least 1% w (percent by weight), relative to the weight of the absorbent, in particular at least 5% w, more in particular at least 8% w, relative to the weight of the absorbent. The absorbent after calcination may contain copper oxide in quantity of at most 100% w, relative to the weight of the absorbent, in particular at most 75% w, more in particular at most 60% w, relative to the weight of the absorbent. The absorbent after calcination may contain copper oxide in a quantity in the range of from 8 to 75% w, relative to the weight of the absorbent, in particular from 15 to 60% w, more in particular from 20 to 50% w, most in particular from 30 to 40% w, relative to the weight of the absorbent.

When the absorbent comprises copper, the absorbent after calcination may contain the additional metal oxide and copper oxide in a mass ratio of metal oxide to copper oxide of at least 0.2, in particular at least 0.5, more in particular at least 0.7. The mass ratio of metal oxide to copper oxide may be at most 10, in particular at most 8, more in particular at most 5. The mass ratio of metal oxide to copper oxide may be in the range of from 0.5 to 10, in particular from 1 to 5, more in particular from 1.2 to 2.5, most in particular from 1.25 to 1.75.

After calcination, the absorbent may or may not be subjected to hydrogen reduction. Typically, hydrogen reduction may be conducted by contacting the absorbent with a hydrogen reduction stream at a temperature in the range of from 150 to 350° C. A suitable hydrogen reduction stream may contain hydrogen in the range of from 0.1 to 10% v (percent by volume) and nitrogen in the range of from 99.9 to 90% v, relative to the total reduction stream. After hydrogen reduction, the absorbent may be subjected to oxygen stabilization. Oxygen stabilization may be conducted by contacting the reduced absorbent at a temperature in the range of 60 to 80° C. with a gas stream containing oxygen in the range of from 0.1 to 10% v and nitrogen in the range of from 99.9 to 90% v, relative to the total stabilization stream.

The absorbent may contain a total amount of the metals (measured as the weight of the metal elements relative to the weight of the absorbent) in a quantity in the range of from 15 to 90% w (percent by weight), in particular from 20 to 85% w, more in particular from 25 to 75% w, measured as the weight of the metal elements relative to the weight of the absorbent.

The support material may be present in the absorbent in a quantity of at least 1% w, relative to the weight of the absorbent, in particular at least 1.5% w, more in particular at least 2% w, relative to the weight of the absorbent. The support material may be present in the absorbent in a quantity of at most 80% w, relative to the weight of the absorbent, in particular at most 50% w, more in particular at most 30% w, relative to the weight of the absorbent, most in particular at most 25% w, relative to the weight of the absorbent. The support material may be present in the absorbent in a quantity in the range of from 5 to 25% w, in particular from 10 to 20% w, relative to the weight of the absorbent.

When the absorbent comprises copper, the absorbent may contain copper in a quantity of at least 1% w (percent by weight), measured as the weight of the copper element relative to the weight of the absorbent, in particular at least 5% w, more in particular more than 8% w, most in particular at least 20% w, measured as the weight of the copper element relative to the weight of the absorbent. The absorbent may contain copper in quantity of at most 85% w, in particular at most 75% w, more in particular at most 60% w, measured as the weight of the copper element relative to the weight of the absorbent. The absorbent may contain copper in a quantity in the range of from 10 to 75% w, in particular from 15 to 60% w, more in particular from 20 to 50% w, most in particular from 25 to 40% w, measured as the weight of the copper element relative to the weight of the absorbent.

When the absorbent comprises copper, the absorbent may contain the additional metal(s) and copper in a ratio of the mass of the additional metal(s) present in the absorbent to the mass of copper present in the absorbent of at least 0.2, in particular at least 0.5, more in particular at least 0.7 (basis the respective elements). The mass ratio of the additional metal(s) to copper may be at most 10, in particular at most 8, more in particular at most 5, same basis. The mass ratio of the additional metal(s) to copper may be in the range of from 0.5 to 10, in particular from 1 to 5, more in particular from 1.2 to 2.5, most in particular from 1.25 to 1.75, same basis.

The sulfur impurities may include, but are not limited to, dihydrogen sulfide, carbonyl sulfide, mercaptans, organic sulfides, and combinations thereof. The mercaptans may include methanethiol or ethanethiol. The organic sulfides may include aromatic sulfides or alkyl sulfides, such as dimethylsulfide. Mercaptans and organic sulfides, in particular organic sulfides, are particularly difficult sulfur impurities to remove from a feed. In the treated feed (i.e., the feed after contact with the absorbent), the quantity of sulfur impurities may be at most 70% w of the total quantity of sulfur impurities present in the untreated feed, preferably at most 35% w, more preferably at most 10% w, on the same basis.

The treated feed is then contacted with the epoxidation catalyst under process conditions sufficient to yield a reaction product comprising an olefin oxide. The following description provides details of a silver-containing epoxidation catalyst, its preparation and its use in an epoxidation process.

The catalyst typically used for the epoxidation of an olefin is a catalyst comprising silver deposited on a carrier. The size and shape of the catalyst is not critical to the invention and may be in the form of chunks, pieces, cylinders, rings, spheres, wagon wheels, tablets, and the like of a size suitable for employment in a fixed bed shell-and-tube heat exchanger reactor vessel, for example from 2 mm to 20 mm.

The carrier may be based on a wide range of materials. Such materials may be natural or artificial inorganic materials and they may include refractory materials, silicon carbide, clays, zeolites, charcoal, and alkaline earth metal carbonates, for example calcium carbonate. Preferred are refractory materials, such as alumina, magnesia, zirconia, silica, and mixtures thereof. The most preferred material is α-alumina. Typically, the carrier comprises at least 85% w, more typically at least 90% w, in particular at least 95% w α-alumina, frequently up to 99.9% w α-alumina, relative to the weight of the carrier. Other components of the α-alumina carrier may comprise, for example, silica, titania, zirconia, alkali metal components, for example sodium and/or potassium components, and/or alkaline earth metal components, for example calcium and/or magnesium components.

The surface area of the carrier may suitably be at least 0.1 m²/g, preferably at least 0.3 m²/g, more preferably at least 0.5 m²/g, and in particular at least 0.6 m²/g, relative to the weight of the carrier; and the surface area may suitably be at most 10 m²/g, preferably at most 6 m²/g, and in particular at most 4 m²/g, relative to the weight of the carrier. “Surface area” as used herein is understood to relate to the surface area as determined by the B.E.T. (Brunauer, Emmett and Teller) method as described in Journal of the American Chemical Society 60 (1938) pp. 309-316. High surface area carriers, in particular when they are alpha alumina carriers optionally comprising in addition silica, alkali metal and/or alkaline earth metal components, provide improved performance and stability of operation.

The water absorption of the carrier may suitably be at least 0.2 g/g, preferably at least 0.25 g/g, more preferably at least 0.3 g/g, most preferably at least 0.35 g/g; and the water absorption may suitably be at most 0.85 g/g, preferably at most 0.7 g/g, more preferably at most 0.65 g/g, most preferably at most 0.6 g/g. The water absorption of the carrier may be in the range of from 0.2 to 0.85 g/g, preferably in the range of from 0.25 to 0.7 g/g, more preferably from 0.3 to 0.65 g/g, most preferably from 0.3 to 0.6 g/g. A higher water absorption may be in favor in view of a more efficient deposition of the metal and promoters, if any, on the carrier by impregnation. However, at a higher water absorption, the carrier, or the catalyst made therefrom, may have lower crush strength. As used herein, water absorption is deemed to have been measured in accordance with ASTM C20, and water absorption is expressed as the weight of the water that can be absorbed into the pores of the carrier, relative to the weight of the carrier.

The preparation of the catalyst comprising silver is known in the art and the known methods are applicable to the preparation of the shaped catalyst particles which may be used in the practice of this invention. Methods of depositing silver on the carrier include impregnating the carrier with a silver compound containing cationic silver and/or complexed silver and performing a reduction to form metallic silver particles. For further description of such methods, reference may be made to U.S. Pat. No. 5,380,697, U.S. Pat. No. 5,739,075, EP-A-266015, and U.S. Pat. No. 6,368,998, which methods are incorporated herein by reference. Suitably, silver dispersions, for example silver sols, may be used to deposit silver on the carrier.

The reduction of cationic silver to metallic silver may be accomplished during a step in which the catalyst is dried, so that the reduction as such does not require a separate process step. This may be the case if the silver containing impregnation solution comprises a reducing agent, for example, an oxalate, a lactate or formaldehyde.

Appreciable catalytic activity may be obtained by employing a silver content of the catalyst of at least 10 g/kg, relative to the weight of the catalyst. Preferably, the catalyst comprises silver in a quantity of from 50 to 500 g/kg, more preferably from 100 to 400 g/kg, for example 105 g/kg, or 120 g/kg, or 190 g/kg, or 250 g/kg, or 350 g/kg, on the same basis. As used herein, unless otherwise specified, the weight of the catalyst is deemed to be the total weight of the catalyst including the weight of the carrier and catalytic components.

The catalyst for use in this invention may comprise a promoter component which comprises an element selected from rhenium, tungsten, molybdenum, chromium, nitrate- or nitrite-forming compounds, and combinations thereof. Preferably the promoter component comprises, as an element, rhenium. The form in which the promoter component may be deposited onto the carrier is not material to the invention. Rhenium, molybdenum, tungsten, chromium or the nitrate- or nitrite-forming compound may suitably be provided as an oxyanion, for example, as a perrhenate, molybdate, tungstate, or nitrate, in salt or acid form.

The promoter component may typically be present in a quantity of at least 0.1 mmole/kg, more typically at least 0.5 mmole/kg, in particular at least 1 mmole/kg, more in particular at least 1.5 mmole/kg, calculated as the total quantity of the element (that is rhenium, tungsten, molybdenum and/or chromium) relative to the weight of the catalyst. The promoter component may be present in a quantity of at most 50 mmole/kg, preferably at most 10 mmole/kg, calculated as the total quantity of the element relative to the weight of the catalyst.

When the catalyst comprises rhenium as the promoter component, the catalyst may preferably comprise a rhenium co-promoter, as a further component deposited on the carrier. Suitably, the rhenium co-promoter may be selected from components comprising an element selected from tungsten, chromium, molybdenum, sulfur, phosphorus, boron, and combinations thereof. Preferably, the rhenium co-promoter is selected from tungsten, chromium, molybdenum, sulfur, and combinations thereof. It is particularly preferred that the rhenium co-promoter comprises, as an element, tungsten and/or sulfur.

The rhenium co-promoter may typically be present in a total quantity of at least 0.1 mmole/kg, more typically at least 0.25 mmole/kg, and preferably at least 0.5 mmole/kg, calculated as the element (i.e. the total of tungsten, chromium, molybdenum, sulfur, phosphorus and/or boron), relative to the weight of the catalyst. The rhenium co-promoter may be present in a total quantity of at most 40 mmole/kg, preferably at most 10 mmole/kg, more preferably at most 5 mmole/kg, on the same basis. The form in which the rhenium co-promoter may be deposited on the carrier is not material to the invention. For example, it may suitably be provided as an oxide or as an oxyanion, for example, as a sulfate, borate or molybdate, in salt or acid form.

The catalyst preferably comprises silver, the promoter component, and a component comprising a further element, deposited on the carrier. Eligible further elements may be selected from the group of nitrogen, fluorine, alkali metals, alkaline earth metals, titanium, hafnium, zirconium, vanadium, thallium, thorium, tantalum, niobium, gallium and germanium and combinations thereof. Preferably the alkali metals are selected from lithium, potassium, rubidium and cesium. Most preferably the alkali metal is lithium, potassium and/or cesium. Preferably the alkaline earth metals are selected from calcium, magnesium and barium. Typically, the further element is present in the catalyst in a total quantity of from 0.01 to 500 mmole/kg, more typically from 0.05 to 100 mmole/kg, calculated as the element on the weight of the catalyst. The further elements may be provided in any form. For example, salts of an alkali metal or an alkaline earth metal are suitable. For example, lithium compounds may be lithium hydroxide or lithium nitrate.

Preferred amounts of the components of the catalysts are, when calculated as the element, relative to the weight of the catalyst:

silver from 10 to 500 g/kg,

rhenium from 0.01 to 50 mmole/kg, if present,

the further element or elements, if present, each from 0.1 to 500 mmole/kg, and,

the rhenium co-promoter from 0.1 to 30 mmole/kg, if present.

As used herein, the quantity of alkali metal present in the catalyst is deemed to be the quantity insofar as it can be extracted from the catalyst with de-ionized water at 100° C. The extraction method involves extracting a 10-gram sample of the catalyst three times by heating it in 20 ml portions of de-ionized water for 5 minutes at 100° C. and determining in the combined extracts the relevant metals by using a known method, for example atomic absorption spectroscopy.

As used herein, the quantity of alkaline earth metal present in the catalyst is deemed to be the quantity insofar as it can be extracted from the catalyst with 10% w nitric acid in de-ionized water at 100° C. The extraction method involves extracting a 10-gram sample of the catalyst by boiling it with a 100 ml portion of 10% w nitric acid for 30 minutes (1 atm., i.e. 101.3 kPa) and determining in the combined extracts the relevant metals by using a known method, for example atomic absorption spectroscopy. Reference is made to U.S. Pat. No. 5,801,259, which is incorporated herein by reference.

Although the present epoxidation process may be carried out in many ways, it is preferred to carry it out as a gas phase process, i.e. a process in which the feed is first contacted in the gas phase with a packed bed of absorbent to yield a treated feed, as described herein, and subsequently the treated gaseous feed is contacted with a packed bed of epoxidation catalyst. Generally the process is carried out as a continuous process.

The reaction feed comprises an olefin and may include any olefin, such as an aromatic olefin, for example styrene, or a di-olefin, whether conjugated or not, for example 1,9-decadiene or 1,3-butadiene. Preferably, the olefin may be a monoolefin, for example 2-butene or isobutene. More preferably, the olefin may be a mono-α-olefin, for example 1-butene or propylene. The most preferred olefin is ethylene. Suitably, mixtures of olefins may be used.

The olefin may be obtained from several sources including, but not limited to, petroleum processing streams such as those generated by a thermal cracker, a catalytic cracker, a hydrocracker or a reformer, natural gas fractions, naphtha, and organic oxygenates such as alcohols. The alcohols are typically derived from the fermentation of various biomaterials including, but not limited to, sugar cane, syrup, beet juice, molasses, and other starch-based materials. An olefin, such as ethylene, derived from an alcohol prepared via a fermentation process can be a particularly troublesome source of impurities, especially sulfur impurities.

The olefin may be present in a quantity of at least 0.5 mole-%, relative to the total feed, in particular at least 1 mole-%, more in particular at least 15 mole-%, most in particular at least 20 mole-%, on the same basis. The olefin may be present in the feed in a quantity of at most 80 mole-%, relative to the total feed, in particular at most 70 mole-%, more in particular at most 60 mole-%, on the same basis.

The feed also contains oxygen as a reactant. The present epoxidation process may be air-based or oxygen-based, see “Kirk-Othmer Encyclopedia of Chemical Technology”, 3^{rd} edition, Volume 9, 1980, pp. 445-447. In the air-based process, air or air enriched with oxygen is employed as the source of the oxidizing agent while in the oxygen-based processes high-purity (at least 95 mole-%) oxygen or very high purity (at least 99.5 mole-%) oxygen is employed as the source of the oxidizing agent. Reference may be made to U.S. Pat. No. 6,040,467, incorporated by reference, for further description of oxygen-based processes. Presently most epoxidation plants are oxygen-based and this is a preferred embodiment of the present invention.

In order to remain outside the flammable regime, the quantity of oxygen in the feed may be lowered as the quantity of the olefin is increased. The actual safe operating ranges depend, along with the feed composition, also on the reaction conditions such as the reaction temperature and the pressure.

Oxygen may be present in a quantity of at least 0.5 mole-%, relative to the total feed, in particular at least 1 mole-%, more in particular at least 2 mole-%, most in particular at least 5 mole-%, relative to the total feed. Oxygen may be present in a quantity of at most 25 mole-%, relative to the total feed, in particular at most 20 mole-%, more in particular at most 15 mole-%, most in particular at most 12 mole-%, relative to the total feed. As used herein, the feed is considered to be the composition which is contacted with the absorbent.

In addition to the olefin and oxygen, the reaction feed may further comprise a saturated hydrocarbon as a dilution gas. The feed may further comprise a reaction modifier, an inert dilution gas, and a recycle gas stream.

The saturated hydrocarbon may be selected from methane, ethane, propane, butane, pentane, hexane, heptane, octane, nonane, decane, undecane, dodecane and mixtures thereof. In particular, the saturated hydrocarbon may be selected from methane, ethane, propane, and mixtures thereof, preferably methane. Saturated hydrocarbons are common dilution gases in an epoxidation process and can be a significant source of impurities in the feed, especially sulfur impurities. Saturated hydrocarbons may be added to the feed in order to increase the oxygen flammability limit.

The saturated hydrocarbon may be present in a quantity of at least 1 mole-%, relative to the total feed, in particular at least 10 mole-%, more in particular at least 20 mole-%, most in particular at least 30 mole-%, on the same basis. The saturated hydrocarbon may be present in the feed in a quantity of at most 80 mole-%, relative to the total feed, in particular at most 75 mole-%, more in particular at most 70 mole-%, most in particular at most 65 mole-%, on the same basis.

It is unexpected that the absorbent can reduce the amount of impurities, especially sulfur impurities, in a feed containing a combination of feed components under the conditions experienced inside the reactor vessel. It is especially unexpected that the absorbent can reduce the amount of impurities in a feed which contains oxygen as a reactant at the elevated oxidation temperatures experienced inside the reactor vessel.

A reaction modifier may be present in the feed for increasing the selectively, suppressing the undesirable oxidation of olefin or olefin oxide to carbon dioxide and water, relative to the desired formation of olefin oxide. Many organic compounds, especially organic halides and organic nitrogen compounds, may be employed as the reaction modifiers. Nitrogen oxides, organic nitro compounds such as nitromethane, nitroethane, and nitropropane, hydrazine, hydroxylamine or ammonia may be employed as well. It is frequently considered that under the operating conditions of olefin epoxidation the nitrogen containing reaction modifiers are precursors of nitrates or nitrites, i.e. they are so-called nitrate- or nitrite-forming compounds (cf. e.g. EP-A-3642 and U.S. Pat. No. 4,822,900, which are incorporated herein by reference).

Organic halides are the preferred reaction modifiers, in particular organic bromides, and more in particular organic chlorides. Preferred organic halides are chlorohydrocarbons or bromohydrocarbons. More preferably they are selected from the group of methyl chloride, ethyl chloride, ethylene dichloride, ethylene dibromide, vinyl chloride or a mixture thereof. Most preferred reaction modifiers are ethyl chloride and ethylene dichloride.

Suitable nitrogen oxides are of the general formula NOₓ wherein x is in the range of from 1 to 2.5, and include for example NO, N₂O₃, N₂O₄, and N₂O₅. Suitable organic nitrogen compounds are nitro compounds, nitroso compounds, amines, nitrates and nitrites, for example nitromethane, 1-nitropropane or 2-nitropropane. In preferred embodiments, nitrate- or nitrite-forming compounds, e.g. nitrogen oxides and/or organic nitrogen compounds, are used together with an organic halide, in particular an organic chloride.

The reaction modifiers are generally effective when used in small quantities in the feed, for example at most 0.1 mole-%, relative to the total feed, for example from 0.01×10⁻⁴ to 0.01 mole-%. In particular when the olefin is ethylene, it is preferred that the reaction modifier is present in the feed in a quantity of from 0.1×10⁻⁴ to 500×10⁻⁴ mole-%, in particular from 0.2×10⁻⁴ to 200×10⁻⁴ mole-%, relative to the total feed.

A recycle gas stream may be used as a feed component in the epoxidation process. The reaction product comprises the olefin oxide, unreacted olefin, unreacted oxygen, reaction modifier, dilution gases, and, optionally, other reaction by-products such as carbon dioxide and water. The reaction product is passed through one or more separation systems, such as an olefin oxide absorber and a carbon dioxide absorber, so the unreacted olefin and oxygen may be recycled to the reactor system. Carbon dioxide is a by-product in the epoxidation process. However, carbon dioxide generally has an adverse effect on the catalyst activity. Typically, a quantity of carbon dioxide in the feed in excess of 25 mole-%, in particular in excess of 10 mole-%, relative to the total feed, is avoided. A quantity of carbon dioxide of less than 3 mole-%, preferably less than 2 mole-%, more preferably less than 1 mole-%, relative to the total feed, may be employed. Under commercial operations, a quantity of carbon dioxide of at least 0.1 mole-%, in particular at least 0.2 mole-%, relative to the total feed, may be present in the feed.

Inert dilution gases, for example nitrogen, helium or argon, may be present in the feed in a quantity of from 30 to 90 mole-%, typically from 40 to 80 mole-%, relative to the total feed.

The epoxidation process is preferably carried out at a reactor inlet pressure in the range of from 1000 to 3500 kPa. “GHSV” or Gas Hourly Space Velocity is the unit volume of gas at normal temperature and pressure (0° C., 1 atm, i.e. 101.3 kPa) passing over one unit volume of packed catalyst per hour. Preferably, when the epoxidation process is a gas phase process involving a packed catalyst bed, the GHSV is in the range of from 1500 to 10000 Nl/(l·h). Preferably, the process is carried out at a work rate in the range of from 0.5 to 10 kmole olefin oxide produced per m³ of catalyst per hour, in particular 0.7 to 8 kmole olefin oxide produced per m³ of catalyst per hour, for example 5 kmole olefin oxide produced per m³ of catalyst per hour. As used herein, the work rate is the amount of the olefin oxide produced per unit volume of catalyst per hour and the selectivity is the molar quantity of the olefin oxide formed relative to the molar quantity of the olefin converted. As used herein, the activity is a measurement of the temperature required to achieve a particular ethylene oxide production level. The lower the temperature, the better the activity.

The olefin oxide produced in the epoxidation process may be converted into a 1,2-diol, a 1,2-diol ether, a 1,2-carbonate, or an alkanolamine. As this invention leads to a more attractive process for the production of the olefin oxide, it concurrently leads to a more attractive process which comprises producing the olefin oxide in accordance with the invention and the subsequent use of the obtained olefin oxide in the manufacture of the 1,2-diol, 1,2-diol ether, 1,2-carbonate, and/or alkanolamine.

The conversion into the 1,2-diol or the 1,2-diol ether may comprise, for example, reacting the olefin oxide with water, suitably using an acidic or a basic catalyst. For example, for making predominantly the 1,2-diol and less 1,2-diol ether, the olefin oxide may be reacted with a ten fold molar excess of water, in a liquid phase reaction in presence of an acid catalyst, e.g. 0.5-1.0% w sulfuric acid, based on the total reaction mixture, at 50-70° C. at 1 bar absolute, or in a gas phase reaction at 130-240° C. and 20-40 bar absolute, preferably in the absence of a catalyst. The presence of such a large quantity of water may favor the selective formation of 1,2-diol and may function as a sink for the reaction exotherm, helping control the reaction temperature. If the proportion of water is lowered, the proportion of 1,2-diol ethers in the reaction mixture is increased. The 1,2-diol ethers thus produced may be a di-ether, tri-ether, tetra-ether or a subsequent ether. Alternative 1,2-diol ethers may be prepared by converting the olefin oxide with an alcohol, in particular a primary alcohol, such as methanol or ethanol, by replacing at least a portion of the water by the alcohol.

The olefin oxide may be converted into the corresponding 1,2-carbonate by reacting it with carbon dioxide. If desired, a 1,2-diol may be prepared by subsequently reacting the 1,2-carbonate with water or an alcohol to form the 1,2-diol. For applicable methods, reference is made to U.S. Pat. No. 6,080,897, which is incorporated herein by reference.

The conversion into the alkanolamine may comprise, for example, reacting the olefin oxide with ammonia. Anhydrous ammonia is typically used to favor the production of monoalkanolamine. For methods applicable in the conversion of the olefin oxide into the alkanolamine, reference may be made to, for example U.S. Pat. No. 4,845,296, which is incorporated herein by reference.

The 1,2-diol and the 1,2-diol ether may be used in a large variety of industrial applications, for example in the fields of food, beverages, tobacco, cosmetics, thermoplastic polymers, curable resin systems, detergents, heat transfer systems, etc. The 1,2-carbonates may be used as a diluent, in particular as a solvent. The alkanolamine may be used, for example, in the treating (“sweetening”) of natural gas.

Unless specified otherwise, the low-molecular weight organic compounds mentioned herein, for example the olefins, 1,2-diols, 1,2-diol ethers, 1,2-carbonates, alkanolamines, and reaction modifiers, have typically at most 40 carbon atoms, more typically at most 20 carbon atoms, in particular at most 10 carbon atoms, more in particular at most 6 carbon atoms. As defined herein, ranges for numbers of carbon atoms (i.e. carbon number) include the numbers specified for the limits of the ranges.

Having generally described the invention, a further understanding may be obtained by reference to the following examples, which are provided for purposes of illustration only and are not intended to be limiting unless otherwise specified.

### EXAMPLES

### Example 1

Absorbent A was prepared by a co-precipitation method which included hydrogen reduction and oxygen stabilization. After calcination, Absorbent A had a content of about 36% w CuO, 48% w ZnO, and 16% w Al₂O₃.

The following is a prophetic co-precipitation method which may be used to prepare the above absorbent. A solution of metal nitrates is prepared by dissolving metal components of aluminum, copper and zinc (in that order) in dilute nitric acid. The amount of the metal components are such as to yield a finished precipitate after calcination of about 36% w CuO; 48% w ZnO; and 16% w Al₂O₃. A soda solution (160-180 g/l) is prepared and transferred to a precipitation vessel. The soda solution is heated to 80° C. The mixed nitrate solution is then added to the soda solution over approximately 2 hours while stirring. During the precipitation process, the temperature is adjusted to keep the temperature at approximately 80° C. The precipitation is stopped once a pH of 8.0 (+0.2) is achieved. The stirring of the slurry is continued for 30 minutes at 80° C. and the pH measured again (the pH can be adjusted, if necessary, by the addition of the soda solution or the nitrate solution). The concentration of the oxide in the slurry is approximately 60 grams of oxide per liter of slurry. The precipitate is then filtered and washed. The precipitate is then dried at a temperature in the range of from 120-150° C. and then calcined at a temperature of 400-500° C. The precipitate is then formed into 5×5 mm tablets.

The tablets are then reduced using diluted hydrogen (0.1 to 10% volume H₂ in N₂) at 190 to 250° C. The reduced tablets are then stabilized using dilute oxygen (0.1 to 10% volume O₂ in N₂) at a maximum temperature of 80° C.

Absorbent A was tested by placing into a stainless steel U-shaped tube of internal diameter 4.8 mm a 4 g sample of Absorbent A that had been ground to a size range of 14-20 mesh. Absorbent A was fixed in the tube by means of glass wool plugs. The tube was placed in a molten metal bath, and was maintained at a temperature of 180° C.

A feedstock consisting of 30% v C₂H₄, 8.0% v O₂, 5.0% v CO₂, 2.5 ppmv ethyl chloride, and balance N₂ was directed through the heated tube containing Absorbent A at a flow rate of 280 cc/min. Also included in the feedstock was dimethylsulfide, the concentration of which was varied from 0.62 to 10 ppmv over the course of the experiment. The sulfur contaminant was introduced into the feedstock by blending a stock gas mixture, which was composed of 49.9 ppmv dimethylsulfide in nitrogen, into the ethylene stream prior to mixing the ethylene with other feed components. The total pressure within the tube was maintained at 210 psig.

The gas exiting the first absorbent containing tube was directed through a second stainless steel U-shaped tube of internal diameter 4.8 mm that contained 0.5 g of catalyst. The catalyst, which consisted of 14.5% w silver and 500 ppmw cesium supported on alpha alumina, was maintained at 230° C. and 210 psig. The catalyst was used to react with and quantify any dimethylsulfide that penetrated through the upstream absorbent bed. After 24 hours, the catalyst tube was removed for chemical analysis.

Subsequently, the catalyst tube was either immediately replaced by a new catalyst tube for a time interval of 24 hours or replaced by an empty tube for a time interval ranging from 24 to 72 hours, which allowed continued exposure of the absorbent to the sulfur-containing feedstock at a known rate. For each catalyst tube removed, the catalyst was crushed to a fine powder, thoroughly mixed, and then analyzed by x-ray photoelectron spectroscopy to quantify the amount of sulfur that had penetrated the upstream absorbent bed and reacted with the catalyst.

For sulfur measurement purposes, a sulfur-containing gas mixture was fed directly through several catalyst samples for a variety of time intervals. Each such sample was analyzed by x-ray photoelectron spectroscopy to quantify the amount of sulfur that had reacted with the catalyst. A standardization curve was constructed that correlated x-ray photoelectron spectroscopy signal intensities with net sulfur exposure. This standardization curve was employed to quantify the amount of the sulfur on the catalyst during each data collection interval of Example 1 and Example 2.

The data for sulfur removal by Absorbent A under these conditions is summarized in Table I below.

### Example 2

Example 2 was conducted in a similar manner to Example 1, except for the following two changes: 1) Absorbent A was maintained at a temperature of 25° C. instead of a temperature of 180° C. as was maintained in Example 1; and 2) Absorbent A was placed in the sulfur-containing ethylene stream upstream from the junction where the ethylene stream is combined with the rest of the feed components, instead of being placed in the fully constituted feed stream as was done in Example 1. In Example 2, the sulfur-ethylene mixture was directed over Absorbent A and then the resulting treated ethylene was combined with the other feedstock components and fed to the catalyst bed. In Example 1, all of the feed components were combined upstream of the Absorbent A bed and the catalyst bed.

The data for sulfur removal by Absorbent A under these conditions is summarized below in Table I.

TABLE I

| | EXAMPLE 1 | EXAMPLE 2 |
|---|---|---|
| Temperature of Absorbent A: | 180° C. | 25° C. |
| Location relative to the oxygen inlet: | upstream | downstream |
| g Sulfur captured per g Absorbent A | 0.68 | 0.01 |
| when 15% breakthrough is exceeded: | | |
| g Sulfur captured per g Absorbent A | 0.88 | 0.03 |
| when 45% breakthrough is exceeded: | | |
| g Sulfur captured per g Absorbent A | 1.10 | 0.06 |
| when 90% breakthrough is exceeded: | | |

*Percent breakthrough is the weight percentage of sulfur fed that was not absorbed by the guard bed

### Example 3

A reactor vessel containing a commercial scale reactor tube having an internal diameter of 21 mm and a length of 12.8 meters (42 feet) was filled with 2903 g of a catalyst (representing a catalyst bed height of about 39 feet) and, on top of the catalyst, 85.9 g of Absorbent A, see description above in Example 1, was added to give an absorbent bed height in the reactor tube of 0.3 meters (1 foot), 2.4% of the length of the reactor tube. Prior to introducing the Absorbent A tablets into the reactor tube, the tablets were heated in air at 500° C. for 1 hour.

The catalyst comprised silver, rhenium, tungsten, and cesium on α-alumina. Reference may be made to U.S. Pat. No. 4,766,105 for preparation methods.

A feed comprising 30 mole-% ethylene, 8.0 mole-% oxygen, 5.0 mole-% carbon dioxide, 4.0 ppmv ethyl chloride, 0.67 ppmv H₂S (dihydrogen sulfide), balance nitrogen, was introduced into the reactor vessel at a GHSV of 2690 Nl/(l·h) basis the catalyst bed. This same flow represents a GHSV of 106,000 Nl/(l·h) basis the absorbent bed. The temperature of the bed was maintained at 230° C.

After 57 hours, the feed was discontinued and the amount of S (sulfur) on the absorbent and the catalyst was determined by x-ray fluorescence (XRF) analysis of bed fractions. Results are provided in Table II. The absorbent bed captured 54% of the sulfur that was absorbed in the reactor over the testing interval.

TABLE II

| | Mass (g) | Sulfur Absorbed (mg) |
|---|---|---|
| Absorbent A Bed | 85.9 | 369 |
| Catalyst Bed | 2903 | 311 |

### Example 4

The following materials were tested: Comparative X which was an inert material comprising a silica-alumina; Comparative Y which was a slaked lime material containing calcium hydroxide and sodium hydroxide; and Absorbent A, described in Example 1. Each material was tested by placing into separate stainless steel U-shaped tubes of internal diameter 4.8 mm a 3.5-6.5 g sample of material that had been ground to a size range of 20-30 mesh. Each material was fixed in the tube in four equal mass fractions separated by glass wool plugs. Each tube was placed in a molten metal bath, and was maintained at a temperature of 180° C.

A feedstock consisting of 30% v C₂H₄, 8.0% v O₂, 5.0% v CO₂, 3 ppmv ethyl chloride, and balance N₂ was directed through each heated tube at a total flow rate of 1 L/min. Also included in the feedstock was dihydrogen sulfide in a concentration of 7.5 ppmv. A total of 0.0141 grams of sulfur was fed into each tube. The sulfur contaminant was introduced into the feedstock by blending a stock gas mixture, which was composed of 204 ppmv dihydrogen sulfide in nitrogen. The total pressure within the tube was maintained at 210 psig.

Each of the four fractions of each bed was analyzed for sulfur content using x-ray fluorescence spectroscopy to determine the amount of sulfur which had been absorbed by each material. The results are summarized below in Table III. Absorption efficiency is the weight percent of sulfur absorbed by the material relative to the total sulfur contacted with the material.

TABLE III

| | Mass (g) | Volume (cc) | Total Sulfur | Absorption |
|---|---|---|---|---|
| | in U-shaped | in U-shaped | absorbed | Effectiveness |
| Material | tube | tube | (g) | (%) |
| Comparative X | 6.5 | 5.2 | 0.00007 | 0.5 |
| Comparative Y | 3.5 | 5.2 | 0.0057 | 40 |
| Absorbent A | 4 | 5.2 | 0.011 | 75 |

What is claimed is:

1. An epoxidation reactor system comprising:
an epoxidation reactor vessel, and
positioned inside the epoxidation reactor vessel, an absorbent comprising a metal having an atomic number of 22 through 44 or 82 and an epoxidation catalyst positioned downstream from the absorbent.

2. The reactor system as claimed in claim 1, wherein the reactor vessel is a shell-and-tube heat exchanger comprising one or more open-ended reactor tubes positioned substantially parallel to the central longitudinal axis of the vessel; wherein the upper ends are connected to a substantially horizontal upper tube plate and the lower ends are connected to a substantially horizontal lower tube plate.

3. The reactor system as claimed in claim 2, wherein the reactor vessel comprises a quantity of reactor tubes in the range of from 1000 to 20000.

4. The reactor system as claimed in claim 1, wherein the absorbent comprises a metal having an atomic number of 22 through 30.

5. The reactor system as claimed in claim 1, wherein the absorbent comprises one or more metals selected from the group consisting of cobalt, chromium, copper, manganese, nickel, and zinc.

6. The reactor system as claimed in claim 1, wherein the absorbent comprises copper and one or more metals having an atomic number of 22 through 44.

7. The reactor system as claimed in claim 1, wherein the absorbent comprises copper and one or more metals selected from the group consisting of manganese, chromium, zinc, and combinations thereof.

8. The reactor system as claimed in claim 7, wherein the absorbent comprises oxides of copper and zinc.

9. The reactor system as claimed in claim 1, wherein the absorbent further comprises a support material selected from the group consisting of alumina, titania, silica, activated carbon, and mixtures thereof.

10. The reactor system as claimed in claim 2, wherein at least a portion of the absorbent is positioned upstream from the one or more reactor tubes.

11. The reactor system as claimed in claim 10, wherein the absorbent is present in the form of a packed bed having a bed height of at least 0.05 m.

12. The reactor system as claimed in claim 2, wherein at least a portion of the absorbent is positioned inside one or more of the reactor tubes.

13. The reactor system as claimed in claim 12, wherein the absorbent is present in the form of a packed bed having a bed height of at least 0.25% of the length of the reactor tube.

14. The reactor system as claimed in claim 12, wherein the absorbent is present in the form of a packed bed having a bed height of at most 20% of the length of the reactor tube.

15. The reactor system as claimed in claim 1, wherein the catalyst comprises silver.

16. The reactor system as claimed in claim 15, wherein silver is present in a quantity in the range of from 50 to 500 g/kg, relative to the weight of the catalyst

17. The reactor system as claimed in claim 15, wherein silver is present in a quantity in the range of from 100 to 400 g/kg, relative to the weight of the catalyst.

18. The reactor system as claimed in claim 15, wherein the catalyst further comprises one or more selectivity enhancing dopants selected from the group consisting of rhenium, molybdenum, tungsten, chromium, nitrate- or nitrite-forming compounds, and combinations thereof.

19. A process for reacting a feed comprising an olefin, oxygen, and one or more impurities, which process comprises:
contacting the feed with an absorbent comprising a metal having an atomic number of 22 through 44 or 82 positioned within an epoxidation reactor vessel to reduce the quantity of the one or more impurities in the feed; and
subsequently contacting the feed with an epoxidation catalyst to yield an olefin oxide.

20. The process as claimed in claim 19, wherein the feed is contacted with the absorbent at a temperature of at least 140° C.

21. The process as claimed in claim 19, wherein the olefin comprises ethylene.

22. The process as claimed in claim 19, wherein the olefin is present in a quantity of at most 80 mole-%, relative to the total feed, relative to the total feed.

23. The process as claimed in claim 19, wherein the one or more impurities comprise one or more sulfur impurities selected from the group consisting of dihydrogen sulfide, carbonyl sulfide, mercaptans, and organic sulfides.

24. The process as claimed in claim 19, wherein oxygen is present in a quantity in the range of from 2 to 15 mole-%, relative to the total feed.

25. The process as claimed in claim 19, wherein the feed further comprises a saturated hydrocarbon selected from the group consisting of methane, ethane, and mixtures thereof.

26. The process as claimed in claim 25, wherein the saturated hydrocarbon is present in a quantity of at most 80 mole-%, relative to the total feed.

27. The process as claimed in claim 19, wherein the feed further comprises a reaction modifier.

28. The process as claimed in claim 19, wherein the absorbent comprises a metal having an atomic number of 22 through 30.

29. The process as claimed in claim 19, wherein the absorbent comprises one or more metals selected from the group consisting of cobalt, chromium, copper, manganese, nickel, and zinc.

30. The process as claimed in claim 19, wherein the absorbent comprises copper and one or more metals having an atomic number of 22 through 44.

31. The process as claimed in claim 19, wherein the absorbent comprises copper and one or more metals selected from the group consisting of manganese, chromium, zinc, and combinations thereof.

32. The process as claimed in claim 31, wherein the absorbent comprises oxides of copper and zinc.

33. The process as claimed in claim 19, wherein the absorbent further comprises a support material selected from the group consisting of alumina, titania, silica, activated carbon, and mixtures thereof.

34. The process as claimed in claim 19, wherein the catalyst comprises silver.

35. The process as claimed in claim 34, wherein the catalyst further comprises one or more selectivity enhancing dopants selected from the group consisting of rhenium, molybdenum, tungsten, chromium, nitrate- or nitrite-forming compounds, and combinations thereof.

36. A process for preparing a 1,2-diol, a 1,2-diol ether, a 1,2-carbonate, or an alkanolamine comprising converting an olefin oxide into the 1,2-diol, the 1,2-diol ether, the 1,2-carbonate, or the alkanolamine wherein the olefin oxide has been prepared by the process as claimed in claim 19.
